SEQUENCE LISTING (46 sequences)

SEQ ID NO: 1 - protein, 5 aa, Artificial Sequence
  Synthetic peptide epitope
ARLKR

SEQ ID NO: 2 - protein, 5 aa, Artificial Sequence
  Synthetic peptide epitope
  MISC_FEATURE (1)..(1): Xaa can be Alanine or Histidine
  MISC_FEATURE (3)..(3): Xaa can be Leucine, Asparagine, Serine, or Phenylalanine
  MISC_FEATURE (4)..(4): Xaa can be Lysine or Asparagine
  MISC_FEATURE (5)..(5): Xaa can be Arginine or Lysine
XRXXX

SEQ ID NO: 3 - protein, 5 aa, Artificial Sequence
  Synthetic peptide epitope
AAGPP

SEQ ID NO: 4 - protein, 5 aa, Artificial Sequence
  Synthetic peptide epitope
KARRP

SEQ ID NO: 5 - protein, 5 aa, Artificial Sequence
  Synthetic peptide epitope
PAGDR

SEQ ID NO: 6 - protein, 5 aa, Artificial Sequence
  Synthetic peptide epitope
RPEGR

SEQ ID NO: 7 - protein, 5 aa, Artificial Sequence
  Synthetic peptide epitope
AGFKG

SEQ ID NO: 8 - protein, 5 aa, Artificial Sequence
  Synthetic peptide epitope
KGFKG

SEQ ID NO: 9 - protein, 5 aa, Artificial Sequence
  Synthetic peptide epitope
PDKEV

SEQ ID NO: 10 - protein, 5 aa, Artificial Sequence
  Synthetic peptide epitope
RPGFG

SEQ ID NO: 11 - protein, 5 aa, Artificial Sequence
  Synthetic peptide epitope
ANPNA

SEQ ID NO: 12 - protein, 5 aa, Artificial Sequence
  Synthetic peptide epitope
KRGSG

SEQ ID NO: 13 - protein, 5 aa, Artificial Sequence
  Synthetic peptide epitope
PGAKG

SEQ ID NO: 14 - protein, 5 aa, Artificial Sequence
  Synthetic peptide epitope
RPSQR

SEQ ID NO: 15 - protein, 5 aa, Artificial Sequence
  Synthetic peptide epitope
APKRG

SEQ ID NO: 16 - protein, 5 aa, Artificial Sequence
  Synthetic peptide epitope
KRPSQ

SEQ ID NO: 17 - protein, 5 aa, Artificial Sequence
  Synthetic peptide epitope
PKARR

SEQ ID NO: 18 - protein, 5 aa, Artificial Sequence
  Synthetic peptide epitope
RPSWG

SEQ ID NO: 19 - protein, 5 aa, Artificial Sequence
  Synthetic peptide epitope
ARHGF

SEQ ID NO: 20 - protein, 5 aa, Artificial Sequence
  Synthetic peptide epitope
LAGPK

SEQ ID NO: 21 - protein, 5 aa, Artificial Sequence
  Synthetic peptide epitope
PKRGS

SEQ ID NO: 22 - protein, 5 aa, Artificial Sequence
  Synthetic peptide epitope
RRPEG

SEQ ID NO: 23 - protein, 5 aa, Artificial Sequence
  Synthetic peptide epitope
FASRG

SEQ ID NO: 24 - protein, 5 aa, Artificial Sequence
  Synthetic peptide epitope
LGPKG

SEQ ID NO: 25 - protein, 5 aa, Artificial Sequence
  Synthetic peptide epitope
PPSQG

SEQ ID NO: 26 - protein, 5 aa, Artificial Sequence
  Synthetic peptide epitope
RSQPR

SEQ ID NO: 27 - protein, 5 aa, Artificial Sequence
  Synthetic peptide epitope
GKWLG

SEQ ID NO: 28 - protein, 5 aa, Artificial Sequence
  Synthetic peptide epitope
LNPSV

SEQ ID NO: 29 - protein, 5 aa, Artificial Sequence
  Synthetic peptide epitope
PSQGK

SEQ ID NO: 30 - protein, 5 aa, Artificial Sequence
  Synthetic peptide epitope
SNKGA

SEQ ID NO: 31 - protein, 5 aa, Artificial Sequence
  Synthetic peptide epitope
GPKGA

SEQ ID NO: 32 - protein, 5 aa, Artificial Sequence
  Synthetic peptide epitope
LPLGS

SEQ ID NO: 33 - protein, 5 aa, Artificial Sequence
  Synthetic peptide epitope
PSWGP

SEQ ID NO: 34 - protein, 5 aa, Artificial Sequence
  Synthetic peptide epitope
SQGKG

SEQ ID NO: 35 - protein, 5 aa, Artificial Sequence
  Synthetic peptide epitope
GPQGA

SEQ ID NO: 36 - protein, 5 aa, Artificial Sequence
  Synthetic peptide epitope
LSGKP

SEQ ID NO: 37 - protein, 5 aa, Artificial Sequence
  Synthetic peptide epitope
QRHGS

SEQ ID NO: 38 - protein, 5 aa, Artificial Sequence
  Synthetic peptide epitope
VHFFK

SEQ ID NO: 39 - protein, 5 aa, Artificial Sequence
  Synthetic peptide epitope
GSNKG

SEQ ID NO: 40 - protein, 5 aa, Artificial Sequence
  Synthetic peptide epitope
LSPRG

SEQ ID NO: 41 - protein, 5 aa, Artificial Sequence
  Synthetic peptide epitope
RGLFG

SEQ ID NO: 42 - protein, 5 aa, Artificial Sequence
  Synthetic peptide epitope
VYLLP

SEQ ID NO: 43 - protein, 5 aa, Artificial Sequence
  Synthetic peptide epitope
HFDLS

SEQ ID NO: 44 - protein, 5 aa, Artificial Sequence
  Synthetic peptide epitope
NKPSK

SEQ ID NO: 45 - protein, 5 aa, Artificial Sequence
  Synthetic peptide epitope
RGSGK

SEQ ID NO: 46 - protein, 5 aa, Artificial Sequence
  Synthetic peptide epitope
AGPKG

(19) United States
(12) Patent Application Publication
(10) Pub. No.: US 2021/0164991 A1
(43) Pub. Date: Jun. 3, 2021

(54) INDUCED COMMON ANTIBODY RESPONSE

(71) Applicant: ARIZONA BOARD OF REGENTS ON BEHALF OF ARIZONA STATE UNIVERSITY, Scottsdale, AZ (US)

(72) Inventors: Stephen JOHNSTON, Tempe, AZ (US); Lu WANG, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 16/954,187

(22) PCT Filed: Jan. 16, 2019
(86) PCT No.: PCT/US2019/013835

§ 371 Date: Jul. 2, 2020

Related U.S. Application Data
(60) Provisional application No. 62/619,645, filed on Jan. 19, 2018.

(51) Int. Cl. G01N 33/68; C12N 7/00; C12N 1/10; C12N 1/20; C07K 14/47

(52) U.S. Cl. CPC G01N 33/6854; C12N 7/00; C07K 14/4702; C12N 1/20; C12N 1/10

(57) ABSTRACT

A method of inducing a generalized immune response, includes administering to a subject an immunologically effective amount of one or more isolated immunogenic peptides comprising an amino acid sequence of SEQ. ID NOs: 1-46, thereby inducing a generalized immune response to infection by a pathogen in a subject. A prophylactic or therapeutic composition for inducing a generalized immune response, includes an immunologically effective amount of one or more isolated immunogenic peptides comprising an amino acid sequence of SEQ. ID NOs: 1-46. A method of distinguishing a subject infected with a pathogen from an uninfected subject, includes detecting an antibody in the subject that selectively binds one or more isolated immunogenic peptides comprising an amino acid sequence of SEQ. ID NOs: 1-46, wherein the presence of the antibody indicates that the subject is infected with the pathogen. A general method to discover broadly protective components for a vaccine is also disclosed.

### CROSS-REFERENCE TO RELATED APPLICATION

This application claims the benefit of priority to U.S. Provisional Application No. 62/619,645, filed Jan. 19, 2018, the entire contents of which are hereby incorporated herein by reference in its entirety.

### STATEMENT OF GOVERNMENT SUPPORT

This invention was made with government support under grant number HSHQDC-15-C-B0008 awarded by DHS and grant number HDTRA1-12-C-0058 awarded by DTRA. The government has certain rights in the invention.

### FIELD OF THE DISCLOSURE

The present disclosure relates to methods of inducing a common antibody response, and, in particular, inducing a common antibody response using a composition comprising one or more immunogenic peptides or nucleic acids coding such that contain common epitopes conserved among diverse pathogens.

### BACKGROUND

Currently, vaccines are developed to target a single specific pathogen. As such, multiple vaccines are often required to be administered to prevent against common infections. Therefore, it would be beneficial to have a vaccine which was able to induce a common antibody response and capable of treating multiple conditions.

### SUMMARY

An infection is managed by both an innate and an adaptive immune response to the pathogen. It is thought that native antibodies present at the time of infection are a component of the innate response and may play a role by retarding the pathogen¹. This delay allows the second arm, the adaptive response, to be activated and evolve to contain the infection². The inventors have made the surprising discovery of a third arm of the antibody response to infection. As disclosed, the inventors found that 12 different pathogens, including viruses, bacteria and eukaryotes, induce a common set of IgG reactivity. This response was discernible using immunosignature technology. Using sera from 405 infected and non-infected people, it was found that almost all the infected samples can be sorted by the pattern from non-infected people.

Based upon these findings, disclosed are methods of inducing a generalized immune response to infection by a pathogen in a subject. In some embodiments, the method includes selecting a subject for treatment that has, or is at risk for developing, an infection by the pathogen; administering to a subject an immunlogically effective amount of one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, thereby inducing a generalized immune response to infection by a pathogen in a subject. In some examples, the one or more isolated immunogenic peptides or nucleic acids encoding them are administered by one or more of an intranasal route, an intravenous route, a topical route, an enteral route, a parenteral route, or a intravitral route.

Also disclosed therapeutic compositions for inducing a generalized immune response to infection by a pathogen in a subject. In some embodiments, a therapeutic composition comprises a immunologically effective amount of one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46; and a carrier. In some embodiments, a disclosed therapeutic composition is used in the manufacture of a medicament for the treatment of an infection from the pathogen of interest.

In additional embodiments, methods of distinguishing a subject infected with a pathogen from a subject not infected are disclosed. In some embodiments, these methods include selecting a subject for treatment that has, or is at risk for developing, an infection by a pathogen and detecting an antibody in the subject that selectively binds to one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, wherein the presence of the antibody indicates that the subject is infected with a pathogen.

In some examples of the disclosed methods and compositions, the pathogen is a bacterial pathogen of interest, such as one that causes Tuberculosis, Borrelia, or Syphilis. In some examples of the disclosed methods and compositions, the pathogen is a viral pathogen of interest or viral infected cells of interest, such as one that causes HBV, Dengue, Flu, or HIV. In some examples of the disclosed methods and compositions, the pathogen is a fungal pathogen of interest, such as one associated with Valley Fever. In some examples of the disclosed methods and compositions, the pathogen is a parasite, such as causing Chagas or Malaria.

The foregoing and other features of the disclosure will become more apparent from the following detailed description, which proceeds with reference to the accompanying figures.

### BRIEF DESCRIPTION OF THE DRAWINGS

FIG. 1 is a diagram of hierarchical clustering of 5 infections showing separation of each disease. 100 peptides are selected for each disease by One-versus-all T-Test comparison. 500 peptides are then combined for use in the clustering. Each disease has its own signature and is different from other diseases.

FIG. 2 is a diagram of whole immunosignature clustering of 7 pathogens versus healthy donor. Pathogens share red label indicated using DI. Healthy donors are blue indicated by ND. Samples are placed row-wise. All 330,000 peptides are shown in column-wise direction. Pathogens taking together can be clustered apart from healthy donor, while the pathogens cannot be differentiated with each other. All pathogens share large group of common signature responsible for this hierarchical clustering result

FIGS. 3A-3C show that using selected peptides can repeat the separation of pathogens as a group to healthy donor. (FIG. 3A) Peptides selected from pair-wise T-Test between each pathogen vs Healthy combined together shows separation between the 2 groups. (FIG. 3B) PCA analysis shows same separation and Component 1 accounts for over 50% of the variance. (FIG. 3C) Using peptides from T-Test between healthy donors with only one pathogen (BPE) can also separate all the pathogens from healthy together

FIGS. 4A and 4B are plots of two sets of sequences blasted against IEDB and plant pathogens. 500 peptides from the common signature is compared with 500 randomly selected peptides. Peptides from the common signature shows more similarity to sequences in IEDB. When compared with plant pathogens, 500 common peptides are less similar to them than randomly selected peptides from the immunosignature.

FIG. 5 is a diagram of a whole Immunosignature clustering of 12 pathogens versus healthy donor. This analysis used different samples from those in FIG. 1, adding Flu, HIV, Tuberculosis, Chagas, VF infections and on a different Immunosignature array with 125,000 peptides to replicate the result as in FIG. 1. The same clustering pattern is produced: the infections can be distinguished from the non-infected, while the pathogens are mixed together with each other.

FIG. 6 is a diagram of showing that cancers cannot be differentiated from healthy using the same method. The cancer antibody repertoire will either appear to be normal or different with equal probability. This indicates the immune system of 50% of the cancer patients are suppressed.

FIGS. 7A and 7B is a plot and table showing analysis of the common signature reveals dominant epitope that is enriched in pathogen space. (FIG. 7A) ARLKR (SEQ ID NO: 1) epitope was identified as the top consensus epitope after analyzing peptides from the common signature. (FIG. 7B) Blast the epitope against the 7 pathogens fund the epitope in most proteomes.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples:

Adjuvant: A vehicle used to enhance antigenicity. Adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages) Immunostimulatory oligonucleotides (such as those including a CpG motif) can also be used as adjuvants (for example see U.S. Pat. No. 6,194,388; U.S. Pat.. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L and 41 BBL.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Amplification: Of a nucleic acid molecule (e.g., a DNA or RNA molecule) refers to use of a technique that increases the number of copies of a nucleic acid molecule in a specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev Immunol 2:239, 1984). Humanized antibodies and fully human antibodies are also known in the art.

Animal: Living multi-cellular vertebrate organisms, a category that includes, for example, mammals and birds. The term mammal includes both human and non-human mammals. Similarly, the term “subject” includes both human and veterinary subjects.

Diagnostic: Identifying the presence or nature of a pathologic condition, such as, but not limited to, an infection with a pathogen. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition, such as prostate cancer, or metastasis.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda , plac, ptrp, ptac (ptrp-lac hybrid promoter) and the like can be used. In one embodiment, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences.

Host cells: Cells in which a vector can be propagated and its DNA expressed. The cell may be prokaryotic or eukaryotic. The cell can be mammalian, such as a human cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Immunogenic composition: A composition comprising an immunogenic polypeptide or a nucleic acid encoding the immunogenic polypeptide disclosed herein. For in vitro use, the immunogenic composition can consist of the isolated nucleic acid, vector including the nucleic acid/or immunogenic peptide. For in vivo use, the immunogenic composition will typically comprise the nucleic acid, vector including the nucleic acid, and or immunogenic polypeptide, in pharmaceutically acceptable carriers, and/or other agents. An immunogenic composition can optionally include an adjuvant, a costimulatory molecule, or a nucleic acid encoding a costimulatory molecule.

Inhibiting or treating a disease: Inhibiting a disease, such as an infection with a pathogen, refers to inhibiting the full development of a disease. “Treatment” refers to a therapeutic intervention that ameliorates a sign or symptom of a disease or pathological condition related to the disease, such as the infection.

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include fluorescent tags, enzymatic linkages, and radioactive isotopes.

Linker sequence: A linker sequence is an amino acid sequence that covalently links two polypeptide domains. Linker sequences can be included in the between the immunogenic peptides disclosed herein to provide rotational freedom to the linked polypeptide domains and thereby to promote proper domain folding and presentation to the MHC. Linker sequences, which are generally between 2 and 25 amino acids in length, are well known in the art.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence, such as a sequence that encodes a disclosed immunogenic polypeptide. Generally, operably linked DNA sequences are contiguous and, where necessary to join two protein-coding regions, in the same reading frame.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition or a cell to achieve a desired effect in a subject being treated. In some examples, a therapeutically effective amount is an immunologically effective amount. For instance, this can be the amount necessary to induce an immune response for prevention or treatment against a specific pathogen. When administered to a subject, a dosage will generally be used that will achieve the desired concentration that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### Description of Several Embodiments

Introduction

As disclosed herein, the inventors have discovered a third arm of the antibody response to infection. As disclosed, the inventors found that 12 different pathogens, including viruses, bacteria and eukaryotes, induce a common set of IgG reactivity. This response was discernible using immunosignature technology which entails profiling sera antibodies on high-density (125-330 k features) peptide arrays. The peptides are chosen from random sequence space to maximize chemical diversity. Using sera from 405 infected and non-infected people it was found that almost all the infected samples can be sorted by pattern from non-infected people. Thus disclosed herein are methods of detecting an infection in a subject, and/or distinguishing an infected subject from a non-infected subject.

As disclosed herein, a signature that separates a single infection type from non-infected consists of both the common signatures and the specific adapted signature. The common signature peptides can be used to separate any other infection from controls. A comparison of the peptides in the common signature to the Immune Epitope Database (IEDB) identified 44 amino acid sequences that are shared between many pathogens in the IEDB and are in the common signature identified. This data indicates that viruses, bacteria and eukaryotes that have evolved to become a human pathogen elicit a common IgG antibody response to a limited number of shared epitopes. This common response may, like the native antibodies, serve to modulate the infection in the early stages until the specific adaptive response matures. Using other collections of Immunosignature peptides, improved informatic techniques and/or additions of other pathogen epitopes to the IEDB collection it may be possible to identify more antigens that could contribute to a broadly protective vaccine.

The B-cells that produce the common signature could be germline cells, as for native antibodies. There are native B cells in higher vertebrates¹. However, they would need to be induced on infection. On the other hand, these B-cells could have been induced by previous infections and are reactivated on a subsequent infection. Isolation and sequencing of these B-cells should resolve this issue.

Using this common signature immunogenic peptides and compositions have been developed to augment the low response, by vaccination, to a level that is more protective. Such a vaccine has broad implication is treatment of pathogenic infection.

Immunogenic Compositions

Disclosed are immunogenic compositions specifically designed to elicit an immune response, such as an antibody response, to a generalized pathogen population. An immunogenic composition, such as disclosed herein is composition useful for stimulating or eliciting a specific immune response (or immunogenic response) in a vertebrate. In some embodiments, the immunogenic response is protective or provides protective immunity against cancer. One specific example of a type of immunogenic composition is a vaccine. For in vitro use, the immunogenic composition can consist of the isolated nucleic acid, vector including the nucleic acid/or immunogenic polypeptide. For in vivo use, the immunogenic composition will typically comprise immunogenic polypeptide(s) and/or the nucleic acids encoding the immunogenic polypeptide(s), such as a vector including the nucleic acid, in pharmaceutically acceptable carriers, and/or other agents. An immunogenic composition can optionally include an adjuvant. The disclosed immunogenic compositions include one or more isolated polypeptides, such as a plurality, that, when administered to a subject, elicit a general immune response to one or more pathogens. In some embodiments, the polypeptides are non-HLA restricted. In some embodiments the polypeptides are HLA restricted, such as HLA-A24, HLA-A1 and HLA-A2 restricted.

In embodiments, an isolated polypeptide that elicits a general immune response to one or more pathogens comprises consists essentially of, and/or consists of, the amino acid sequence set forth as ARLKR (SEQ. ID NO: 1). In embodiments, an isolated polypeptide that elicits a general immune response to one or more pathogens comprises consists essentially of, and/or consists of, the amino acid sequence set forth as X₁RX₂X₃X₄ (SEQ. ID NO: 2), wherein X₁ is Alanine or Histidine, X₂ is Leucine, Asparagine, Serine, or Phenylalanine, X₃ Lysine or Asparagine, and X₄ is Arginine or Lysine. In embodiments, an isolated polypeptide that elicits a general immune response to one or more pathogens comprises consists essentially of, and/or consists of, the amino acid sequence set forth as one of AAGPP (SEQ. ID NO: 3), KARRP (SEQ. ID NO: 4), PAGDR (SEQ. ID NO: 5), RPEGR (SEQ. ID NO: 6), AGFKG (SEQ. ID NO: 7), KGFKG (SEQ. ID NO: 8), PDKEV (SEQ. ID NO: 9), RPGFG (SEQ. ID NO: 10), ANPNA (SEQ. ID NO: 11), KRGSG (SEQ. ID NO: 12), PGAKG (SEQ. ID NO: 13), RPSQR (SEQ. ID NO: 14), APKRG (SEQ. ID NO: 15), KRPSQ (SEQ. ID NO: 16), PKARR (SEQ. ID NO: 17), RPSWG (SEQ. ID NO: 18), ARHGF (SEQ. ID NO: 19), LAGPK (SEQ. ID NO: 20), PKRGS (SEQ. ID NO: 21), RRPEG (SEQ. ID NO: 22), FASRG (SEQ. ID NO: 23), LGPKG (SEQ. ID NO: 24), PPSQG (SEQ. ID NO: 25), RSQPR (SEQ. ID NO: 26), GKWLG (SEQ. ID NO: 27), LNPSV (SEQ. ID NO: 28), PSQGK (SEQ. ID NO: 29), SNKGA (SEQ. ID NO: 30), GPKGA (SEQ. ID NO: 31), LPLGS (SEQ. ID NO: 32), PSWGP (SEQ. ID NO: 33), SQGKG (SEQ. ID NO: 34), GPQGA (SEQ. ID NO: 35), LSGKP (SEQ. ID NO: 36), QRHGS (SEQ. ID NO: 37), VHFFK (SEQ. ID NO: 38), GSNKG (SEQ. ID NO: 39), LSPRG (SEQ. ID NO: 40), RGLFG (SEQ. ID NO: 41), VYLLP (SEQ. ID NO: 42), HFDLS (SEQ. ID NO: 43), NKPSK (SEQ. ID NO: 44), RGSGK (SEQ. ID NO: 45), AGPKG (SEQ. ID NO: 46).

In some embodiments, a disclosed composition includes one or more of the polypeptides set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, SEQ. ID NO: 46, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, or all 46 of SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, and/or SEQ. ID NO: 46, in any combination.

In some embodiments the pathogen sequences are imbedded in longer sequences to enhance their immunogenicity. For example to provide CD4 help or targeting to a cellular compartment.

. In some embodiments, the pathogen of is a bacterial pathogen of interest. In some embodiments, the bacterial pathogen of interest is Tuberculosis, Borrelia, Malaria or Syphilis. In some embodiments, the pathogen is a viral pathogen of interest or viral infected cells of interest. In some embodiments, the viral pathogen of interest is HBV, Dengue, Flu, or HIV. In some embodiments, the pathogen is a fungal pathogen of interest. In some embodiments, the fungal pathogen of interests is Valley Fever. In some embodiments, the pathogen is a parasite. In some embodiments, the parasite is T. cruzi.

The disclosed isolated polypeptides include synthetic embodiments of polypeptides described herein. In addition, analogs (non-peptide organic molecules), derivatives (chemically functionalized polypeptide molecules obtained starting with the disclosed polypeptide sequences) and variants (homologs) of these polypeptides can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified polypeptides, and optionally having other desirable properties. For example, peptide sequences with lengths exceeding 19 amino acids, may be reduced in length by 1, 2, 3, 4, 5, 6 or 7 amino acids from either the amine end, carboxyl end or both ends of the of the peptide sequence. In another example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring Amino groups of the polypeptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the polypeptide side chains may be converted to C₁-C₁₆ alkoxy or to a C₁-C₁₆ ester using well-recognized techniques. Phenyl and phenolic rings of the polypeptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the polypeptide side chains can be extended to homologous C₂-C₄ alkylenes. Thiols can be protected with any one of a number of well-recognized protecting groups, such as acetamide groups. Those skilled in the art will also recognize methods for introducing cyclic structures into the polypeptides of this invention to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the polypeptide backbone and component amino acid side chains, resulting in such peptido- and organomimetics of an immunogenic polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

In embodiments, an immunogenic polypeptide is included in a fusion protein. For example, any and all of the immunogenic polypeptides included in an immunogenic composition, including a plurality of immunogenic polypeptides, can be in the form of a fusion protein. Thus, the fusion protein can include an immunogenic polypeptide and a second heterologous moiety, such as a myc protein, an enzyme or a carrier (such as a hepatitis carrier protein or bovine serum albumin) covalently linked to the immunogenic polypeptide. A second heterologous moiety can be covalently or non-covalently linked to the immunogenic polypeptide. The immunogenic polypeptides can be included in a fusion protein and can also include heterologous sequences. Thus, in several specific non-limiting examples, one or more of the immunogenic polypeptides are included in a fusion polypeptide, for example a fusion of an immunogenic polypeptide with six sequential histidine residues, a β-galactosidase amino acid sequence, or an immunoglobulin amino acid sequence. The immunogenic polypeptides can also be covalently linked to a carrier. Suitable carriers include, but are not limited to, a hepatitis B small envelope protein HBsAg. This protein has the capacity to self-assemble into aggregates and can form viral-like particles. The preparation of HBsAg is well documented; see for example European Patent Application Publication No. EP-A-0 226 846, European Patent Application Publication No. EP-A-0 299 108 and PCT Publication No. WO 01/117554, and the amino acid sequence disclosed, for example, in Tiollais et al, Nature, 317: 489, 1985, and European Patent Publication No. EP-A-0 278 940, and PCT Publication No. WO 91/14703, all of which are incorporated herein by reference.

A fusion polypeptide can optionally include repetitions of one or more of any of the immunogenic polypeptides disclosed herein. In one specific, non-limiting example, the fusion polypeptide includes two, three, four, five, or up to ten repetitions of a single immunogenic polypeptide. In another example, the fusion polypeptide can optionally include two or more different immunogenic polypeptides disclosed herein. In one specific, non-limiting example, the fusion polypeptide includes two, three, four, five, ten or more different immunogenic polypeptides. A linker sequence can optionally be included between the immunogenic polypeptides.

In some embodiments, two or more different disclosed immunogenic polypeptides can be included on a polypeptide, such as an immunogenic molecule. For example, 2-20 or more different immunogenic polypeptides can be included in the polypeptide, such as 2, 3, 4, 5, 6, 7, 8, 9, 10, 11, 12, 13, 14, 15, 16, 17, 18, 19, 20 or more different immunogenic polypeptides. The different immunogenic polypeptides can be separated by a linking molecule, for example polypeptide linkers, or a molecular scaffold.

The compositions described herein can include varying concentrations of each immunogenic polypeptide in a plurality of immunogenic polypeptides.

The immunogenic polypeptides can be covalently linked to a carrier, which is an immunogenic macromolecule to which an antigenic molecule can be bound. When bound to a carrier, the bound polypeptide becomes more immunogenic. Carriers are chosen to increase the immunogenicity of the bound molecule and/or to elicit higher titers of antibodies against the carrier which are diagnostically, analytically, and/or therapeutically beneficial. Covalent linking of a molecule to a carrier can confer enhanced immunogenicity and T cell dependence (see Pozsgay et al , PNAS 96:5194-97, 1999; Lee et al , J. Immunol. 116: 1711-18, 1976; Dintzis et al , PNAS 73:3671-75, 1976). Useful carriers include polymeric carriers, which can be natural (for example, polysaccharides, polypeptides or proteins from bacteria or viruses), semi-synthetic or synthetic materials containing one or more functional groups to which a reactant moiety can be attached. Bacterial products and viral proteins (such as hepatitis B surface antigen and core antigen) can also be used as carriers, as well as proteins from higher organisms such as keyhole limpet hemocyanin, horseshoe crab hemocyanin, edestin, mammalian serum albumins, and mammalian immunoglobulins. Additional bacterial products for use as carriers include bacterial wall proteins and other products (for example, streptococcal or staphylococcal cell walls and lipopolysaccharide (LPS)).

Nucleic acids encoding one or more of the immunogenic polypeptides are envisioned. These polynucleotides include DNA, cDNA and RNA sequences which encode the polypeptide(s) of interest. Nucleic acid molecules encoding these polypeptides can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same polypeptide.

Nucleic acid sequences encoding one or more of the immunogenic polypeptides can be prepared by any suitable method including, for example, cloning of appropriate sequences or by direct chemical synthesis by methods such as the phosphotriester method of Narang et al, Meth. Enzymol. 68:90-99, 1979; the phosphodiester method of Brown et al, Meth. Enzymol. 68: 109-151, 1979; the diethylphosphoramidite method of Beaucage et al, Tetra. Lett. 22: 1859-1862, 1981 ; the solid phase phosphoramidite triester method described by Beaucage & Caruthers, Tetra. Letts. 22(20): 1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

Exemplary nucleic acids including sequences encoding one or more of the immunogenic polypeptides disclosed herein can be prepared by cloning techniques. Examples of appropriate cloning and sequencing techniques, and instructions sufficient to direct persons of skill through cloning are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Once the nucleic acids encoding one or more of the immunogenic polypeptides are isolated and cloned, the protein can be expressed in a recombinantly engineered cell such as bacteria, plant, yeast, insect and mammalian cells using a suitable expression vector. One or more DNA sequences encoding one or more immunogenic polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding one or more of the immunogenic polypeptides can be operatively linked to expression control sequences (e.g., a promoter). An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e. , ATG) in front of a protein-encoding gene, splicing signal for introns, maintenance of the correct reading frame of that gene to permit proper translation of mRNA, and stop codons.

The polynucleotide sequences encoding one or more of the immunogenic polypeptides can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art.

Biologically functional viral and plasmid DNA vectors capable of expression and replication in a host are known in the art.

In embodiments, the immunogenic composition is a vaccine. A vaccine is a pharmaceutical composition that elicits a prophylactic or therapeutic immune response in a subject. In some cases, the immune response is a protective response. Typically, a vaccine elicits an antigen-specific immune response to an antigen such as an antigen on the surface of a pathogen, for example a bacterial or a viral pathogen.

Therapeutic Formulations

The immunogenic compositions disclosed herein may be included in pharmaceutical compositions (including therapeutic and prophylactic formulations), and may be combined together with one or more pharmaceutically acceptable vehicles and, optionally, other therapeutic ingredients, such as adjuvants.

Such pharmaceutical compositions can be administered to subjects by a variety of administration modes, including by intramuscular, subcutaneous, intravenous, intra-atrial, intra-articular, intraperitoneal, parenteral routes oral, rectal, intranasal, intrapulmonary, or transdermal delivery, or by topical delivery to other surfaces.

In certain embodiments an immunogenic composition is formulated for use in the manufacture of a medicament for the treatment of an infection from the pathogen of interest. In certain embodiments an immunogenic composition is formulated for use as medicament for the treatment of an infection from the pathogen of interest. In certain embodiments an immunogenic composition is formulated is formulated for intranasal, intravenous, topical, enteral, parenteral, or intravitral administration.

To formulate a pharmaceutical composition, the immunogenic compositions can be combined with various pharmaceutically acceptable additives, as well as a base or vehicle for dispersion of the immunogenic compositions. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, TWEEN® 80), solubility enhancing agents (for example, cyclodextrins and derivatives thereof), stabilizers (for example, serum albumin), and reducing agents (for example, glutathione) can be included.

Adjuvants, such as aluminum hydroxide (for example, AMPHOGEL®, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. In embodiments, a immunogenic composition includes Complete Freund's Adjuvant (CFA), gardiquimod and Poly(I:C).

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The immunogenic compositions can be dispersed in a base or vehicle, which can include a hydrophilic compound having a capacity to disperse the immunogenic composition, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface. The immunogenic composition can be combined with the base or vehicle according to a variety of methods, and release of the immunogenic composition can be by diffusion, disintegration of the vehicle, or associated formation of water channels. In some circumstances, the immunogenic composition is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43: 1-5, 1991), and dispersed in a biocompatible dispersing medium, which yields sustained delivery and biological activity over a protracted time. The immunogenic compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the immunogenic compositions can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the immunogenic compositions can be brought about by including in the composition an agent which delays absorption, for example, monostearate salts and gelatin.

In certain embodiments, the immunogenic compositions can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various immunogenic compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the immonogenic compostion and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body. Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble polypeptides (U.S. Pat. No. 4,675,189).

The pharmaceutical compositions of the disclosure typically are sterile and stable under conditions of manufacture, storage and use. Sterile solutions can be prepared by incorporating the immunogenic compositions in the required amount in an appropriate solvent with one or a combination of ingredients enumerated herein, as required, followed by filtered sterilization. Generally, dispersions are prepared by incorporating the immunogenic composition and/or other biologically active agent into a sterile vehicle that contains a basic dispersion medium and the required other ingredients from those enumerated herein. In the case of sterile powders, methods of preparation include vacuum drying and freeze-drying which yields a powder of the immunogenic composition plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

Methods of Treatment

The immunogenic compositions disclosed herein (including immunogenic polypeptides), or nucleic acids encoding the immunogenic polypeptides, polynucleotides encoding such polypeptides and vectors comprising the polynucleotides, can be used in methods of generating or eliciting a generalized immune response to a wide range of pathogens, such as bacterial and/or viral pathogens.

In certain embodiments, the method is a method of inducing a generalized immune response to infection by a pathogen in a subject, comprising: selecting a subject for treatment that has, or is at risk for developing, an infection by a pathogen; administering to a subject a therapeutically effective amount of one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, inducing a generalized immune response to infection by a pathogen in a subject.. In certain embodiments, the one or more isolated immunogenic peptides are administered by one or more of an intranasal route, an intravenous route, a topical route, an enteral route, a parenteral route, or a intravitral route. In certain embodiments, the pathogen of is a bacterial pathogen of interest. In certain embodiments, the bacterial pathogen of interest is Tuberculosis, Borrelia, or Syphilis. In certain embodiments, the pathogen is a viral pathogen of interest or viral infected cells of interest. In certain embodiments, the viral pathogen of interest is HBV, Dengue, Flu, or HIV. In certain embodiments, the pathogen is a fungal pathogen of interest. In certain embodiments, the fungal pathogen of interest is Coccidiodes. In certain embodiments, the pathogen is a parasite. In certain embodiments, the parasite is T. cruzi or Plasmodium.

In several embodiments, the methods include administering to a subject with an effective amount, such as an immunologically effective dose, of one or more of the immunogenic compositions disclosed in order to generate an immune response. The methods can include selecting a subject in need of treatment, such as a subject that has, is suspected of having, or is predisposed to an infection. An immune response is a response of a cell of the immune system, such as a B-cell, T-cell, macrophage or peripheral blood mononuclear cell, to a stimulus. An immune response can include any cell of the body involved in a host defense response. An immune response includes, but is not limited to, an adaptive immune response or inflammation. In some examples, an immune response is stimulated by administering to a subject a vaccine and/or disclosed immunogenic composition.

In exemplary applications, the immunogenic compositions are administered to a subject having a disease, such as an infection with a pathogen, in an amount sufficient to raise an immune response to cells expressing the antigens targeted by the immunogenic composition. Amounts effective for this use will depend upon the severity of the disease, the general state of the patient's health, and the robustness of the patient's immune system. In one example, a therapeutically effective amount of the compound is that which provides either subjective relief of a symptom(s) or an objectively identifiable improvement as noted by the clinician or other qualified observer.

In accordance with the various treatment methods of the disclosure, the immunogenic composition can be delivered to a subject in a manner consistent with conventional methodologies associated with management of the disorder for which treatment or prevention is sought. In accordance with the disclosure herein, a prophylactically or therapeutically effective amount of the immunogenic composition and/or other biologically active agent is administered to a subject in need of such treatment for a time and under conditions sufficient to prevent, inhibit, and/or ameliorate a selected disease or condition or one or more symptom(s) thereof, such as infection.

Typical subjects intended for treatment with the compositions and methods of the present disclosure include humans, as well as non-human primates and other animals. To design the broadly effective vaccine for a different species than humans, eg canines, it may require screening various infection and non-infection sera from that species To identify subjects for prophylaxis or treatment according to the methods of the disclosure, accepted screening methods are employed to determine risk factors associated with a targeted or suspected disease of as discussed herein, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods, which are available and well known in the art to detect and/or characterize disease-associated markers. These and other routine methods allow the clinician to select patients in need of therapy using the methods and pharmaceutical compositions of the disclosure. In accordance with these methods and principles, immunogenic compositions and/or other biologically active agent can be administered according to the teachings herein as an independent prophylaxis or treatment program, or as a follow-up, adjunct or coordinate treatment regimen to other treatments, including surgery, vaccination, immunotherapy, hormone treatment, and the like.

The immunogenic compositions can be used in coordinate vaccination protocols or combinatorial formulations. In certain embodiments, novel combinatorial immunogenic compositions and coordinate immunization protocols employ separate immunogens or formulations, each directed toward eliciting a desired immune response. The separate immunogens disclosed herein can be combined in a polyvalent immunogenic composition administered to a subject in a single immunization step, or they can be administered separately (in monovalent immunogenic compositions) in a coordinate immunization protocol.

The administration of the immunogenic compositions of the disclosure can be for either prophylactic or therapeutic purpose. When provided prophylactically, the immunogenic composition is provided in advance of any symptom. The prophylactic administration of the immunogenic composition serves to prevent or ameliorate any progression on the disease. When provided therapeutically, the immunogenic composition is provided at (or shortly after) the onset of a symptom of disease. For prophylactic and therapeutic purposes, the immunogenic compositions can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the immunogenic composition can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the immunogenic composition (for example, amounts that are effective to elicit a desired immune response or alleviate one or more symptoms of a targeted disease). In alternative embodiments, an effective amount or effective dose of the immunogenic compositions may simply inhibit or enhance one or more selected biological activities correlated with a disease or condition, as set forth herein, for either therapeutic or diagnostic purposes.

The actual dosage of the immunogenic compositions will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the immunogenic compositions for eliciting the desired activity or biological response in the subject. Dosage regimens can be adjusted to provide an optimum prophylactic or therapeutic response. A therapeutically effective amount is a quantity of a specific substance (for example, this may be the amount of a disclosed immunogenic composition useful in increasing resistance to, preventing, ameliorating, and/or treating cancer, such as medullary thyroid carcinoma) sufficient to achieve a desired effect in a subject being treated without causing a substantial cytotoxic effect in the subject. For example, a therapeutically effective amount of composition can vary from about 0.01 mg/kg body weight to about 1 g/kg body weight. When administered to a subject, a dosage will generally be used that will achieve target concentrations shown to achieve a desired in vivo effect. A therapeutically effective amount is also one in which any toxic or detrimental side effects of the immunogenic composition and/or other biologically active agent is outweighed in clinical terms by therapeutically beneficial effects. A non-limiting range for a therapeutically effective amount of a the immunogenic composition and/or other biologically active agent within the methods and formulations of the disclosure is about 0.01 mg/kg body weight to about 10 mg/kg body weight, such as about 0.05 mg/kg to about 5 mg/kg body weight, or about 0.2 mg/kg to about 2 mg/kg body weight.

Upon administration of a immunogenic composition of the disclosure (for example, via injection, aerosol, oral, topical or other route), the immune system of the subject typically responds to the immunogenic composition by producing T cells capable of expanding and reacting to the specific antigenic epitopes presented by the immunogenic composition. Such a response signifies that an immunologically effective dose of the immunogenic composition was delivered. An immunologically effective dosage can be achieved by single or multiple administrations (including, for example, multiple administrations per day), daily, or weekly administrations. For each particular subject, specific dosage regimens can be evaluated and adjusted over time according to the individual need and professional judgment of the person administering or supervising the administration of the immunogenic composition. In some embodiments, the T cell response, as measured by ELISPOT, tetramer staining or intracellular cytokine staining of a subject administered the compositions of the disclosure will be determined in the context of evaluating effective dosages/immunization protocols. In some instances it will be sufficient to assess the percentage of antigen specific T cells and their phenotype via ELISPOT or intracellular cytokine staining. Decisions as to whether to administer booster inoculations and/or to change the amount of the composition administered to the individual can be at least partially based on the ELISPOT data, tetramer staining data or intracellular cytokine staining data.

Dosage can be varied by the attending clinician to maintain a desired concentration. Higher or lower concentrations can be selected based on the mode of delivery. Dosage can also be adjusted based on the release rate of the administered formulation.

These immunogenic compositions can be used for active immunization, and for preparation of immune antibodies. The immunogenic compositions are composed of non-toxic components, suitable for infants, children of all ages, and adults.

Kits are also provided. In one embodiment, these kits include a container or formulation that contains one or more of the immunogenic compositions described herein. In one example, this component is formulated in a pharmaceutical preparation for delivery to a subject. The immunogenic composition is optionally contained in a bulk dispensing container or unit or multi-unit dosage form. Optional dispensing means can be provided. Packaging materials optionally include a label or instruction indicating for what treatment purposes and/or in what manner the pharmaceutical agent packaged therewith can be used.

The immunogenic composition of this disclosure can be employed to generate antibodies that recognize the antigens disclosed herein and the antigen from which the disclosed antigen was derived. The methods include administering to a subject immunogenic composition including a disclosed antigen or administering to the subject a polynucleotide encoding a disclosed antigen to generate antibodies that recognize the disclosed antigen. The subject employed in this embodiment is one typically employed for antibody production. Mammals, such as, rodents, rabbits, goats, sheep, etc., are preferred.

The antibodies generated can be either polyclonal or monoclonal antibodies. Polyclonal antibodies are raised by injecting (for example subcutaneous or intramuscular injection) antigenic polypeptides into a suitable animal (for example, a mouse or a rabbit). The antibodies are then obtained from blood samples taken from the animal. The techniques used to produce polyclonal antibodies are extensively described in the literature. Polyclonal antibodies produced by the subjects can be further purified, for example, by binding to and elution from a matrix that is bound with the polypeptide against which the antibodies were raised. Those of skill in the art will know of various standard techniques for purification and/or concentration of polyclonal, as well as monoclonal, antibodies. Monoclonal antibodies can also be generated using techniques known in the art.

Synthesis of Polypeptides

The polypeptides used in the disclosed immunogenic compositions can be made by any method available in the art, for example synthesized using solid-phase polypeptide synthesis techniques familiar to those in the art, including Fmoc chemistry, or purification of polypeptides from recombinant prokaryotic or eukaryotic sources.

The disclosed immunogenic compositions can be prepared by cloning techniques. Examples of appropriate cloning and sequencing techniques and instructions sufficient to direct persons of skill through many cloning exercises are found in Sambrook et al, Molecular Cloning: A Laboratory Manual (2nd Ed.), Vols. 1-3, Cold Spring Harbor Laboratory (1989), Berger and Kimmel (eds.), Guide to Molecular Cloning Techniques, Academic Press, Inc., San Diego Calif. (1987) or Ausubel et al. (eds.), Current Protocols in Molecular Biology, Greene Publishing and Wiley-Interscience, N.Y. (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH® laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), INVITROGEN™ (San Diego, Calif.) and Applied Biosystems (Foster City, Calif.), as well as many other commercial sources known to one of skill.

Peptides for the disclosed immunogenic compositions may be produced, for example by chemical synthesis by any of a number of manual or automated methods of synthesis known in the art. In addition, polypeptides that form all or part of a hetero-bifunctional ligand can be produced synthetically. For example, solid phase polypeptide synthesis (SPPS) is carried out on a 0.25 millimole (mmole) scale using an Applied Biosystems Model 43 IA Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3 -tetramethyluronium hexafluorophosphate/ hydroxybenzotriazole (HBTU/HOBT) and using p-hydroxymethylphenoxymethylpolystyrene (HMP) or Sasrin resin for carboxyl-terminus acids or Rink amide resin for carboxyl-terminus amides. Fmoc-derivatized amino acids are prepared from the appropriate precursor amino acids by tritylation and triphenylmethanol in trifluoroacetic acid, followed by Fmoc derivitization as described by Atherton et al. Solid Phase Peptide Synthesis, IRL Press: Oxford, 1989.

Methods of Detecting Infection

Disclosed is a method of distinguishing a subject infected with a pathogen from a subject not so infected, comprising: selecting a subject that has, or is at risk for developing, an infection by a pathogen; detecting an antibody in the subject that selectively binds to one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, wherein the presence of the antibody indicates that the subject is infected with the pathogen.

### EXAMPLES

The following example is put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

An infection is managed by both an innate and an adaptive immune response to the pathogen. It is thought that native antibodies present at the time of infection are a component of the innate response and may play a role by retarding the pathogen¹. This delay allows the second arm, the adaptive response, to be activated and evolve to contain the infection². As disclosed herein, the inventors have discovered a third arm of the antibody response to infection. As disclosed, the inventors found that 12 different pathogens, including viruses, bacteria and eukaryotes, induce a common set of IgG reactivity. This response was discernible using immunosignature technology which entails profiling sera antibodies on high-density (125-330 k features) peptide arrays^{3,4}. The peptides are chosen from random sequence space to maximize chemical diversity. Using sera from 405 infected and non-infected people it was found that almost all the infected samples can be sorted by pattern from non-infected people.

A signature that separates a single infection type from non-infected consists of both the common signatures and the specific adapted signature. The common signature peptides can be used to separate any other infection from controls. In addition, a common signature is not evident in comparison of 4 cancer types to non-cancer subjects, indicating that this signature was pathogen dependent. A comparison of the peptides in the common signature to the Immune Epitope Database (IEDB) identified 44 amino acid sequences that are shared between many pathogens in the IEDB and are in the common signature identified⁵. This data indicates that viruses, bacteria and eukaryotes that have evolved to become a human pathogen elicit a common IgG antibody response to a limited number of shared epitopes. This common response may, like the native antibodies, serve to modulate the infection in the early stages until the specific adaptive response matures.

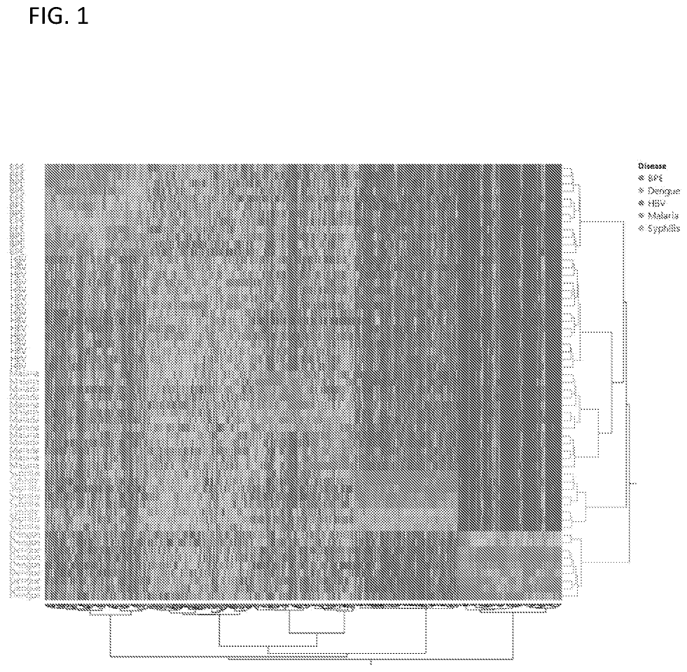

The immunosignature diagnostic platform has been shown to separate the immune responses of a variety of infections from non-infected sera samples, as well as different infections from each other⁶⁻¹². We first demonstrated that the samples we used (Table 1) were also distinguishable on this platform. In FIG. 1, the samples from 5 different infections (BPE, HBV, Dengue, Malaria and Syphilis) were readily distinguished from each other using 500 peptides from the array as a classifier. These peptides were chosen based on their ability to distinguish each infection from the others.

TABLE 1
Samples cohort used in this study.

| Group | Count |
|---|---|
| Borrelia | 8 |
| BPE | 12 |
| Dengue | 9 |
| HBV | 15 |
| Malaria | 13 |
| ND | 32 |
| Syphllis | 8 |
| WNV | 21 |
| Total | 118 |

Seven types of infections along with the normal donor control group were used in this study, with a total sample size of 118.

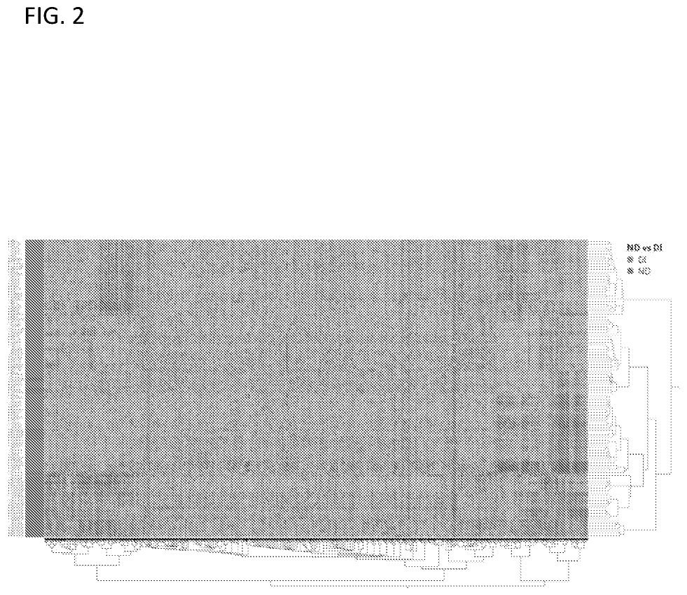

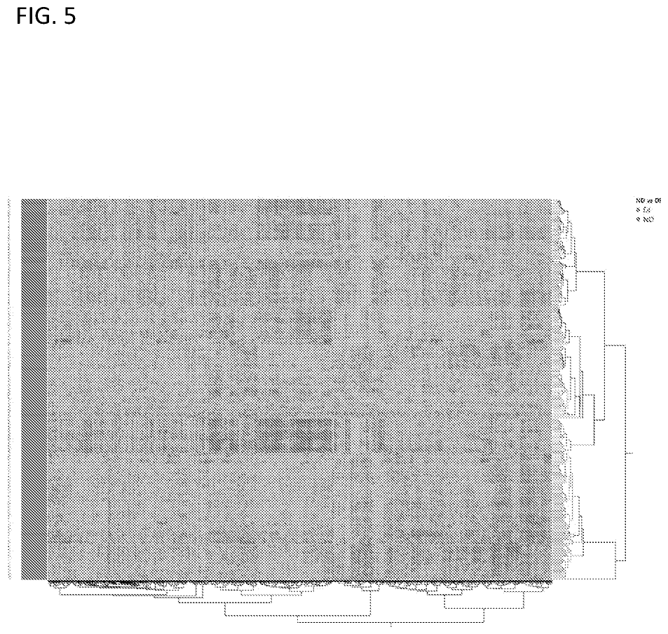

The same array data was reanalyzed without separation based on infection type. All 8 sample sets in Table 1 were included. Two-way hierarchical clustering of the whole immunosignature with 330,000 features was performed. The result of this clustering (FIG. 2) shows that most of the non-infection donors (blue label ND) can be differentiated from the 7 pathogens (red label DI) while the infection samples did not fall into obvious groupings by type of infection. To test the robustness of this observation, the same type of analysis was performed including different samples of the 8 groups in Table 1, adding 5 different infection types (Flu, HIV, Tuberculosis, T. cruzi, Coccidioides (a fungus)) and using a different array format containing 125,000 different peptides. As evident in FIG. 5, most of the 12 different types of infection samples clustered separately from the non-infection samples.

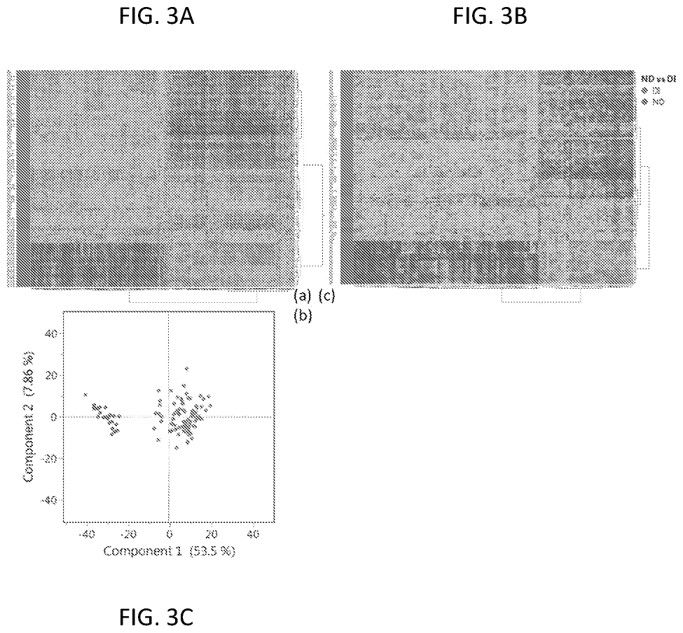

This analysis implies that very different infections elicit antibodies that bind the same peptides on the array. To test this concept from another angle each infection sample set was individually compared to the non-infection group and selected the top 100 peptides (by p-value) for each comparison. Of the 700 peptides selected in this manner, 200 peptides appeared in at least two pathogens. These sequences were pooled and two-way hierarchical clustering was performed for the 7 infections and the non-infection samples. The results are presented in FIG. 3A, showing that these peptides can also be used to separate all infections from non-infection samples. Principle component analysis (FIG. 3B) of this data shows that the first component accounts for over 50% of the variance and using only one component can repeat the same separation result as the clustering.

The implication from the results in FIG. 3A, 3B is that a signature distinguishing any infection from non-infection will be composed of a common and a specific signature. To test this prediction, the 100 peptides chosen that distinguished BPE from non-infection were used as the basis to cluster the other 6 infection groups from non-infection. As shown in FIG. 3C, even though these peptides were not chosen against the other six infections, they were very efficient in making the separation between them and the non-infection group. These data support the concept that there is a common set of IgG antibodies elicited by infections.

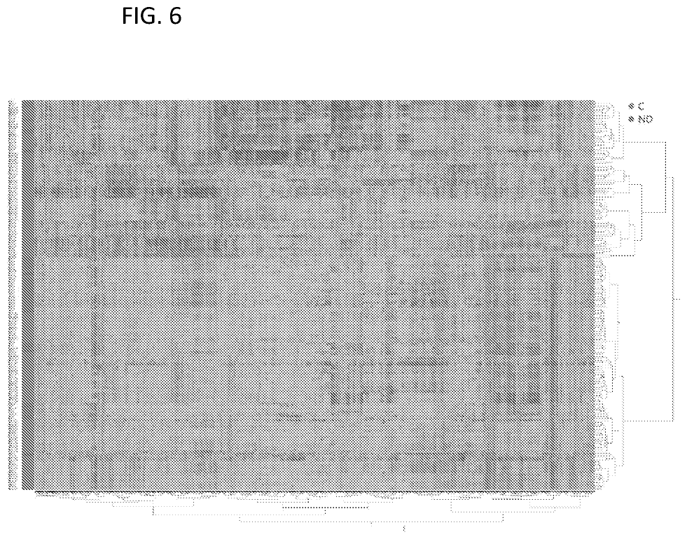

One possibility is that any disease would elicit a common set of antibodies. For example, there are many different types of cancer and they might also elicit a common signature, possibly the same as by infections. To test this, the immunosignatures of 4 different cancers (breast, brain, multiple myeloma and pancreatic) were analyzed in the same manner as we had for the infection samples. As shown in FIG. 6, there was no clear clustering of cancer versus non-cancer samples.

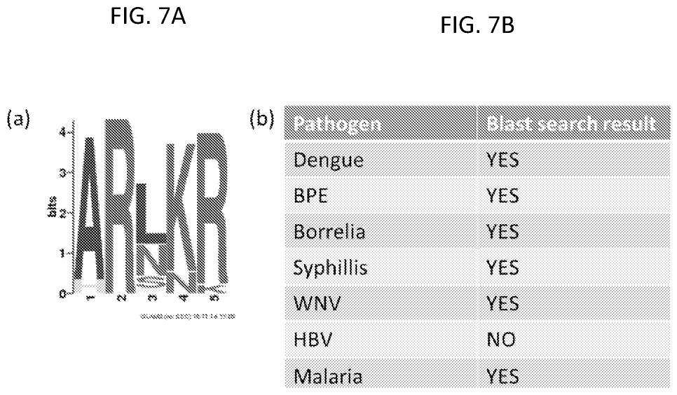

A common signature would imply that there are common epitopes in diverse pathogens that elicit an antibody response. The 330,000 peptides on the array used are on average 12aa long and represent approximately 50% of 5mer peptide space. The implication from the common signature is that these peptides would be related to actual pathogen protein sequences. Two approaches were used to test this. First, the common signature was searched to identify series of enriched pentamers using methods described in Richer et. al¹³. The enriched pentamers were then analyzed in GLAM2 to identify consensus epitopes¹⁴. One dominant epitope, ARLKR (SEQ ID NO: 1), was found (FIG. 7A). This linear epitope was present in 6 of the 7 pathogens used, with hepatitis B virus the exception (FIG. 7B). A second approach was to divide all the peptide sequences in the IEBD into pentamers. The IEDB is a database of verified epitopes in infections. A list of the top 2000 recurrent pentamers from the IEDB was compared to the peptides in the common signature. Forty four pentamers were identified (Table 2). These peptides are presumably at least part of the link between the immune response to infection and the common signature.

TABLE 2
List of the identified enriched epitopes from IEDB. The top
2000 occurring epitopes from IEDB are extracted and tested
on immunosignature. 44 epitopes are identified to be enriched.

| | | | |
|---|---|---|---|
| AAGPP | KARRP | PAGDR | RPEGR |
| AGFKG | KGFKG | PDKEV | RPGFG |
| ANPNA | KRGSG | PGAKG | RPSQR |
| APKRG | KRPSQ | PKARR | RPSWG |
| ARHGF | LAGPK | PKRGS | RRPEG |
| FASRG | LGPKG | PPSQG | RSQPR |
| GKWLG | LNPSV | PSQGK | SNKGA |
| GPKGA | LPLGS | PSWGP | SQGKG |
| GPQGA | LSGKP | QRHGS | VHFFK |
| GSNKG | LSPRG | RGLFG | VYLLP |
| HFDLS | NKPSK | RGSGK | AGPKG |

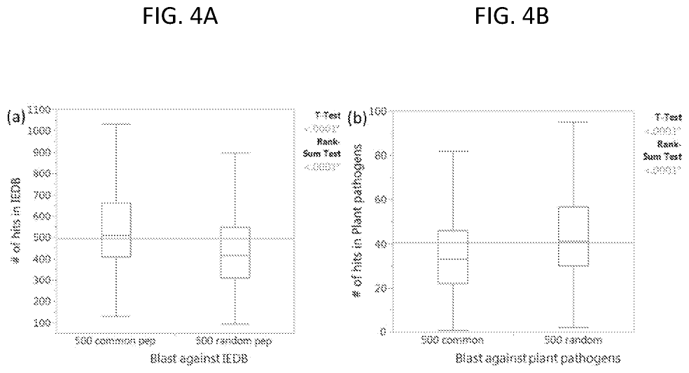

It was further hypothesized that the common signature is the product of the proteomes of diverse pathogens being constrained by the human immune system. If so, one would predict that plant pathogens would not exhibit the same constraints ^{15,16}. To test this, 500 sequences from the common signature with the highest p-values and 500 randomly picked peptides from the array not in the common signature were analyzed. Each set was blasted against the IEDB peptides. As shown in FIG. 4A, the common signature peptides had significantly more hits than the random peptides. This implies that the common signature peptides resemble the IEBD epitopes more than other peptides on the array. The same type of analysis was repeated but blasting against a plant pathogen database¹³. Interestingly, the common signature peptides were significantly less similar to the plant proteins than random peptides. This may reflect that the plant proteome is also under sequence constraints, but different than from antibodies, due to interactions with plant hosts.

Other researchers have noted cross reactive antibodies. Natural antibodies, defined as having germline or near germline variable sequences, bind a wide variety of proteins¹⁸, but are not induced on infection. Usually they are IgM class. In contrast, the common signature antibodies are IgG and are only in infected people. Others have noted cross reactive IgG antibodies^{19,20}. For example, using protein arrays of Yersina pestis, Urlich and co-workers found significant cross reactivity with sera from other gram-negative infections²¹. In at least one example it was proposed to be caused by reaction to conserved proteins across the gram-negative bacteria. While it is possible there is overlap between previous array based cross reactivity and the common signature this is unlikely. The common signature is only approximately 2-fold above the signal in non-infected people, where the adaptive, pathogen specific signal is usually 10-100 fold higher. The immunosignature assay is 10-100× more sensitive than ELISA-type assays⁴. This level of sensitivity is probably necessary to recognize the common signature.

The B-cells that produce the common signature could be germline cells, as for native antibodies^{1,22}. There are native B cells in higher vertebrates¹. However, they would need to be induced on infection. On the other hand, these B-cells could have been induced by previous infections and are reactivated on a subsequent infection. Isolation and sequencing of these B-cells should resolve this issue.

The existence of the common signature, and the common epitopes across most human pathogens that may induce them, has interesting evolutionary implications. One idea is that any persistent human pathogen must have these common epitopes. The antibodies comprising the common signature would constrain the infection enough to allow the host to mount a protective response. It would be beneficial for the pathogen so as to not kill the host²³. In the simplest terms, to evolve to be a human pathogen the organism would have to produce the common signature epitopes. If not, it would kill the host too quickly. The implication is that new, highly lethal pathogens from other hosts may not have the common signature epitopes.

Finally, would this common signature have any clinical value? We note that the level of these antibodies is low relative to the adaptive response. The samples used in this study were from infected people with clinical symptoms so the common signature was not fully protective, though it may have moderated the infection. However, it may be possible to augment the low response, by vaccination, to a level that is more protective. Such a vaccine could have broad value.

Materials. Human sera samples exposed to various pathogens were used. Table 1 shows the total cohort used in this study Immunosignature arrays are manufactured in batches of 312. Each array is in situ synthesized, and consists of 330,000 random-sequence peptides with average length of 12 amino acids. Among these controls are single and double amino acid missense sequences, designed to identify improper sequence synthesis. Also, 250 blank spots are used to estimate local background and spatial variations in global background signals.

Immunosignature assay. Sample buffer contains 3% BSA in 1×PBST, pH 7.3. Secondary incubation buffer contains 0.75% Casein in 1× PBST with 0.05% Tween20. Serum samples in 50:50 glycerol were diluted into sample buffer at ratio of 1:1500, then incubated on Immunosignature array with volume of 150 ul for a final concentration of 1:750. Incubation was 1h at 37° C. with rotation. Arrays were washed 3 times with 1× PBST and rinsed 3 times with ddH₂O. 4 nM secondary anti-IgG antibodies conjugated with Alexa-Fluor 555 (Life Technologies, St. Louis, Mo.) was added to the secondary incubation buffer and then added onto entire Immunosignature microarray for a final volume of 2.5 ml to detect the primary antibody binding in the serum. The incubation is 1h at 37° C. with gentle agitation, then slides were rinsed with blocking buffer, then washed 3× with 1× PBST and 3× ddH₂O then dried. Slides were then scanned at 555 nm with Innoscan 910 scanner at 1.0 um resolution to acquire the image. Feature intensities were extracted using the GenePix Pro 6.0 software (Molecular Devices, Santa Clara, Calif.).

Statistics and Analysis. Analysis was performed using the JMP software (SAS Institute Inc.), R (CRAN repository) and python. Raw data is fetched from each GPR file output by GenePix and normalized to the median before analysis. Whole Immunosignature clustering is performed using all data points for all samples using the hierarchical clustering method. Ward is the distance measure between the samples (columns in heatmaps) and the peptides (rows in heatmaps). Two-tail Student's T-Test is used for feature selection, cutoff is set at either the top 50 or 100 peptides with the best p-value from T-Test. For each set of t-test, the p-value is controlled to be <1/330,000, allowing at most one false positive in 330,000 parallel comparison.

Epitope identification. The algorithm used to identify significant epitopes is described in detail in ¹³. Top 1000 peptides from T-Test obtained by comparing normal samples (control) versus all infected (case) samples are used to identify the epitopes. Epitopes are restricted to 5-mer sequences, ungapped. Once significant epitopes are identified, GLAM2 (at web domain meme-suite.org/tools/glam2) from the MEME suite software is used to identify the consensus^{14,24}.

BLAST searches. BLAST (Basic Local Alignment Search) was used to identify matches in the pathogen proteomes. BLASTP by NCBI via web interference is used with default parameters other than not adjusted for short input sequences(the automatic adjustment for short input sequences yields search parameters that are still too relaxed for sequences as short as 5 amino acids), hitlist size=100, gapcosts=15 for existence and =2 for extension. Matrix is set to be PAM30 and word size is at 2. Expect threshold is set at 10\hat{ }10 to ensure we will have desired number of output. Entrez Query is set with “all[filter] NOT predicted[title] NOT hypothetical[tide]” to remove predicted and hypothetical proteins. Note that here the E-value is not important, because the input sequence is short, so we will always hit sequences by chance, which is the definition of E-value. RefSeq database is used as the target database for BLASTP because of better annotation and less redundancy²⁵. The sequences from the 7 pathogens in the RefSeq database are used in this experiment. Query search against IEDB is performed by finding the exact match of putative conserved sequences (obtained empirically) in the database. BLAST search to identify enrichment of the sequences in the RefSeq database is performed using the BLASTP suite as described above, against all RedSeq proteins. The enrichment is measured by counting the number of unique hits in bacteria and eukaryote and get the percentage of output from bacteria and virus. This information is generated from the BLAST results page from the taxonomy report. Blast search against IEDB and plant pathogens in FIG. 6 is performed by using the blast command line program. For each input peptide, the number of matched sequences is recorded. Then group-wise comparison is performed between the 500 peptides from the disease common signature and 500 randomly selected peptides by T-Test and non-parametric tests. Plant pathogen database is retrieved from Comprehensive Phytopathogen Genomics Resource¹⁷, containing 82 pathogens.

### REFERENCES

References cited herein, and hereby specifically incorporated herein by reference.

1 Ochsenbein, A. F. et al. Control of early viral and bacterial distribution and disease by natural antibodies. Science 286, 2156-2159 (1999).

2 Medzhitov, R. Recognition of microorganisms and activation of the immune response. Nature 449, 819-826, doi:10.1038/nature06246 (2007).

3 Stafford, P. et al. Physical characterization of the ‘Immunosignaturing Effect’. Molecular & Cellular Proteomics, doi:10.1074/mcp.M111.011593 (2012).

4 Sykes, K. F., Legutki, J. B. & Stafford, P. Immunosignaturing: a critical review. Trends in Biotechnology (2012).

5 Vita, R. et al. The immune epitope database (IEDB) 3.0. Nucleic acids research 43, D405-D412 (2014).

6 Donnell, B., Maurer, A., Papandreou-Suppappola, A. & Stafford, P. Time-Frequency Analysis of Peptide Microarray Data: Application to Brain Cancer Immunosignatures. Cancer Informatics, 219-233, doi: 10.4137/CIN.S17285 (2015).

7 Navalkar, K. et al. Application of immunosignatures to diagnosis of Valley Fever. Clinical and Vaccine Immunology in press (2014).

8 Stafford, P., Cichacz, Z., Woodbury, N. W. & Johnston, S. A Immunosignature system for diagnosis of cancer. Proceedings of the National Academy of Sciences 111, E3072-E3080 (2014).

9 Johnston, S. A., Thamm, D. H. & Legutki, J. B. The immunosignature of canine lymphoma: characterization and diagnostic application. BMC cancer 14, 657 (2014).

10 Restrepo, L., Stafford, P. & Johnston, S. A. Feasibility of an early Alzheimer's disease immunosignature diagnostic test. Journal of Neuroimmunology, doi:doi:10.1016/j.jneuroim.2012.09.014 (2012).

11 Restrepo, L., Stafford, P., Magee, D. M. & Johnston, S. A. Application of immunosignatures to the assessment of Alzheimer's disease. Annals of Neurology, 5-18, doi:doi:10.1002/ana.22405 (2011).

12 Legutki, J. B., Magee, D. M., Stafford, P. & Johnston, S. A. A general method for characterization of humoral immunity induced by a vaccine or infection. Vaccine 28, 4529-4537 (2010).

13 Richer, J., Johnston, S. A. & Stafford, P. Epitope Identification from Fixed-complexity Random-sequence Peptide Microarrays. Molecular & Cellular Proteomics 14, 136-147, doi:10.1074/mcp.M114.043513 (2015).

14 Bailey, T. L. et al. MEME SUITE: tools for motif discovery and searching. Nucleic acids research 37, W202-208, doi:10.1093/nar/gkp335 (2009).

15 Király, L., Kunstler, A., Bacsó, R., Hafez, Y. & Király, Z. Similarities and differences in plant and animal immune systems—what is inhibiting pathogens? Acta Phytopathologica et Entomologica Hungarica 48, 187-205, doi:10.1556/APhyt.48.2013.2.1 (2013).

16 Jones, J. D. & Dangl, J. L. The plant immune system. Nature 444, 323-329, doi:10.1038/nature05286 (2006).

17 Hamilton, J. P. et al. The Comprehensive Phytopathogen Genomics Resource: a web-based resource for data-mining plant pathogen genomes. Database: the journal of biological databases and curation 2011, bar053, doi:10.1093/database/bar053 (2011).

18 Notkins, A. L. Polyreactivity of antibody molecules. Trends in immunology 25, 174-179 (2004).

19 Cywes-Bentley, C. et al. Antibody to a conserved antigenic target is protective against diverse prokaryotic and eukaryotic pathogens. Proceedings of the National Academy of Sciences 110, E2209-E2218 (2013).

20 Warter, L., Appanna, R. & Fink, K. Human poly-and cross-reactive anti-viral antibodies and their impact on protection and pathology. Immunologic research 53, 148-161 (2012).

21 Keasey, S. L. et al. Extensive antibody cross-reactivity among infectious gram-negative bacteria revealed by proteome microarray analysis. Molecular & cellular proteomics: MCP 8, 924-935, doi:10.1074/mcp.M800213-MCP200 (2009).

22 Zhou, Z.-H. et al. The Broad Antibacterial Activity of the Natural Antibody Repertoire Is Due to Polyreactive Antibodies. Cell Host and Microbe 1, 51-61 (2007).

23 Cressler, C. E., McLEOD, D. V., Rozins, C., Van Den Hoogen, J. & Day, T. The adaptive evolution of virulence: a review of theoretical predictions and empirical tests. Parasitology 143, 915-930 (2016).

24 Frith, M. C., Saunders, N. F., Kobe, B. & Bailey, T. L. Discovering sequence motifs with arbitrary insertions and deletions. PLoS computational biology 4, e1000071, doi:10.1371/journal.pcbi.1000071 (2008).

25 Pruitt, K. D., Tatusova, T. & Maglott, D. R. NCBI Reference Sequence (RefSeq): a curated non-redundant sequence database of genomes, transcripts and proteins. Nucleic acids research 33, D501-504, doi:10.1093/nar/gki025 (2005).

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

1. A method of inducing a generalized immune response to infection by a pathogen in a subject, comprising:
selecting a subject for treatment that has, or is at risk for developing, an infection by a pathogen;
administering to a subject a immunologically effective amount of one or more isolated immunogenic peptides or nucleic acid encoding the one or more isolated immunogenic peptides wherein the one or more isolated immunogenic peptides comprises an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, thereby inducing a generalized immune response to infection by a pathogen in a subject.

2. The method of claim 1, wherein the one or more isolated immunogenic peptides or nucleic acid encoding said one or more isolated immunogenic peptides are administered by one or more of an intranasal route, an intravenous route, a topical route, an enteral route, a parenteral route, or a intravitral route.

3. The method of claim 1, wherein the pathogen is a bacterial pathogen.

4. The method of claim 3, wherein the bacterial pathogen is Tuberculosis, Borrelia, Malaria or Syphilis.

5. The method of claim 1, wherein the pathogen is a viral pathogen or viral infected cells.

6. The method of claim 5, wherein the viral pathogen of interest is hepatitis B virus (HBV), Dengue, Flu, or human immunodeficiency virus (HIV).

7. The method of claim 1, wherein the pathogen is a fungal pathogen.

8. The method of claim 7, wherein the fungal pathogen is Valley Fever.

9. The method of claim 1, wherein the pathogen is a parasite.

10. The method of claim 9, wherein the parasite is Trypanosoma cruzi or Plasmodium.

11. A therapeutic or prophylactic composition for inducing a generalized immune response to infection by a pathogen in a subject, comprising:
a therapeutically effective amount of one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46; and
a carrier.

12. The composition of claim 11, further comprising a therapeutically effective amount of an antibiotic for the pathogen.

13. The composition of claim 11, wherein the pathogen is a bacterial pathogen.

14. The composition of claim 13, wherein the bacterial pathogen is Tuberculosis, Borrelia, or Syphilis.

15. The composition of claim 11, wherein the pathogen is a viral pathogen or viral infected cells.

16. The composition of claim 15, wherein the viral pathogen is hepatitis B virus (HBV), Dengue, Flu, or human immunodeficiency virus (HIV).

17. The composition of claim 11, wherein the pathogen is a fungal pathogen.

18. The composition of claim 17, wherein the fungal pathogen is Valley Fever.

19. The composition of claim 11, wherein the pathogen is a parasite.

20. The composition of claim 19, wherein the parasite is Trypanosoma cruzi or Plasmodium.

21. The composition of claim 11, for use in the manufacture of a medicament for the treatment or prevention of an infection from the pathogen.

22. The composition of claim 11, wherein the composition is formulated for intranasal, intravenous, topical, enteral, parenteral, or intravitral administration.

23. A method of distinguishing a subject infected with a pathogen from a subject not so infected, comprising:
selecting a subject for treatment that has, or is at risk for developing, an infection by a pathogen;
detecting an antibody in the subject that selectively binds to one or more isolated immunogenic peptides comprising an amino acid sequence set forth as SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, SEQ. ID NO: 21, SEQ. ID NO: 22, SEQ. ID NO: 23, SEQ. ID NO: 24, SEQ. ID NO: 25, SEQ. ID NO: 26, SEQ. ID NO: 27, SEQ. ID NO: 28, SEQ. ID NO: 29, SEQ. ID NO: 30, SEQ. ID NO: 31, SEQ. ID NO: 32, SEQ. ID NO: 33, SEQ. ID NO: 34, SEQ. ID NO: 35, SEQ. ID NO: 36, SEQ. ID NO: 37, SEQ. ID NO: 38, SEQ. ID NO: 39, SEQ. ID NO: 40, SEQ. ID NO: 41, SEQ. ID NO: 42, SEQ. ID NO: 43, SEQ. ID NO: 44, SEQ. ID NO: 45, or SEQ. ID NO: 46, wherein the presence of the antibody indicates that the subject is infected with the pathogen.

24. (canceled)
